(19) United States
(12) Patent Application Publication
(10) Pub. No.: US 2026/0355004 A1
(43) Pub. Date: Nov. 12, 2026

(54) EXTERNAL MENSTRUAL CONTAINERS AND FLUID COLLECTION SYSTEMS INCLUDING THE SAME

(71) Applicant: PUREWICK CORPORATION, Covington, GA (US)

(72) Inventors: Danielle Glickstein, Atlanta, GA (US); Harsimran Minhas, Marietta, GA (US)

(21) Appl. No.: 19/111,921

(22) PCT Filed: Sep. 21, 2022
(86) PCT No.: PCT/US2022/044208

§ 371 Date: Mar. 14, 2025

(51) Int. Cl. A61F 5/455

(52) U.S. Cl. CPC A61F 5/455

(57) ABSTRACT

An example external menstrual container (e.g., bag, cup, etc.) includes at least one fluid impermeable layer (102) defining a container chamber (104) that is configured to receive one or more menstrual fluids (e.g., blood, vaginal fluid, mucosal tissue, cervical mucus, etc.). The fluid impermeable layer of the external menstrual container includes a top side (106) configured to abut a vaginal region of the individual and a bottom side (108) opposite the top side. The top side of the fluid impermeable layer may be configured to allow menstrual fluids to flow from the vaginal region of the individual into the container chamber. The bottom side is configured to be attached to and/or abut a fluid collection assembly.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

In an embodiment, an external menstrual container is disclosed. The external menstrual container includes at least one fluid impermeable layer defining a container chamber. The at least one fluid impermeable layer includes a top side configured to abut a vaginal region of an individual. The top side defines a bag opening configured to allow menstrual fluids to flow from the vaginal region into the container chamber. The at least one fluid impermeable layer also includes a bottom side opposite the top side, the bottom side at least one of configured to be attached to a fluid collection assembly or is concave and configured to abut the fluid collection assembly.

In an embodiment, a fluid collection system is disclosed. The fluid collection system includes a fluid collection assembly. The fluid collection assembly includes a fluid impermeable barrier at least defining a chamber, an opening, and a fluid outlet. The fluid collection assembly also includes at least one porous material disposed in the chamber and extending across the opening. The fluid collection system also includes an external menstrual container. The external menstrual container includes at least one fluid impermeable layer defining a container chamber. The at least one fluid impermeable layer includes a top side configured to abut a vaginal region of an individual. The top side defines a bag opening configured to allow menstrual fluids to flow from the vaginal region into the container chamber. The at least one fluid impermeable layer also includes a bottom side opposite the top side. The bottom side is at least one of configured to be attached to the fluid collection assembly, attached to the fluid collection assembly, or abutting the fluid collection assembly.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a menstrual container, according to an embodiment.

FIG. 1B is a cross-sectional view of the menstrual container taken along plane 1B-1B, according to an embodiment.

FIG. 2 is a cross-sectional schematic of a menstrual container including at least one porous material disposed in the container chamber therein, according to an embodiment.

FIG. 3 is an isometric view of a menstrual container that includes one or more wings that are configured to directly attach the menstrual container to the individual, according to an embodiment.

FIG. 4 is an isometric view of a menstrual container that includes one or more wings that are configured to directly attach the menstrual container to a fluid collection assembly, according to an embodiment.

FIG. 5A is a side view of a fluid collection assembly, according to an embodiment.

FIG. 5B is a cross-sectional schematic of the fluid collection assembly taken along plane 5B-5B shown in FIG. 5A.

FIG. 5C is a cross-sectional schematic of a fluid collection system including the fluid collection assembly with a menstrual container attached thereto, according to an embodiment.

FIG. 6 is a cross-sectional schematic of a fluid collection system including a fluid collection assembly and a menstrual container, according to an embodiment.

FIG. 7 is a cross-sectional schematic view of a fluid collection system disposed on a female individual, according to an embodiment.

FIG. 8 is a block diagram of a fluid collection system for fluid collection, according to an embodiment.

### DETAILED DESCRIPTION

Embodiments are directed to external menstrual containers, fluid collection systems including the same, and methods of using the same. An example external menstrual container (e.g., bag, cup, etc.) includes at least one fluid impermeable layer defining a container chamber that is configured to receive one or more menstrual fluids (e.g., blood, vaginal fluid, mucosal tissue, cervical mucus, etc.). The fluid impermeable layer of the external menstrual container includes a top side configured to abut a vaginal region of the individual and a bottom side opposite the top side. The top side of the fluid impermeable layer may be configured to allow menstrual fluids to flow from the vaginal region of the individual into the container chamber. The bottom side is configured to be attached to and/or abut a fluid collection assembly.

The fluid collection assembly that is configured to be used with the external menstrual containers may include a fluid impermeable barrier defining a chamber, an opening, and a fluid outlet. The fluid collection assembly may also include at least one porous material disposed in the chamber. The fluid collection assembly may further include at least one conduit that is in fluid communication with the chamber via the fluid outlet. The conduit may be attached, either directly or indirectly, to a fluid storage container and a vacuum source.

The external menstrual container may be attached to or abut the fluid collection assembly to form a fluid collection system. During use, the fluid collection system is positioned on the individual such that the bag opening of the external menstrual container is positioned adjacent to the vaginal opening of the individual and a portion of the opening of the fluid collection assembly is positioned adjacent to the urethral opening of the individual. Such positioning of the fluid collection system allows the menstrual fluids discharged by the individual to be received by the external menstrual container instead of the porous material of the fluid collection assembly and allows the bodily fluids (e.g., urine) discharged by the urethral opening to be received into the porous material. The bodily fluids received by the porous material may be removed from the chamber of the fluid collection assembly using the conduit and disposed in the fluid storage container. For example, the vacuum source may apply a suction to the chamber that facilitates removal of the bodily fluids from the chamber.

The external menstrual container allows the fluid collection assembly to be use more efficiently when the individual is menstruating. For example, merely disposing the fluid collection assembly without the external menstrual container causes the porous material to receive the menstrual fluids discharged by the vaginal opening. However, the menstrual fluids received into the porous material may obstruct the pores of the porous material which, in turn, inhibits flow of the bodily fluids through the porous material. For example, the blood in the menstrual fluids may clot thereby permanently obstructing the pores of the porous material. The menstrual fluids also exhibit a greater viscosity of the menstrual fluids relative to the bodily fluids causes the menstrual fluids to remain in the porous material longer, both of which inhibits flow of the bodily fluids through the porous material. The menstrual fluids inhibiting the flow of bodily fluids through the porous material may cause the fluid collection assembly to leak or otherwise fail to receive the bodily fluids discharged from the urethral opening and may decrease the amount of time that fluid collection assembly may be used. However, the external menstrual containers disclosed herein capture the menstrual fluids while preventing or at least inhibiting menstrual fluids from entering the porous material. As such, the external menstrual containers allow the fluid collection assembly to be used with a menstruating individual without compromising the efficiency thereof.

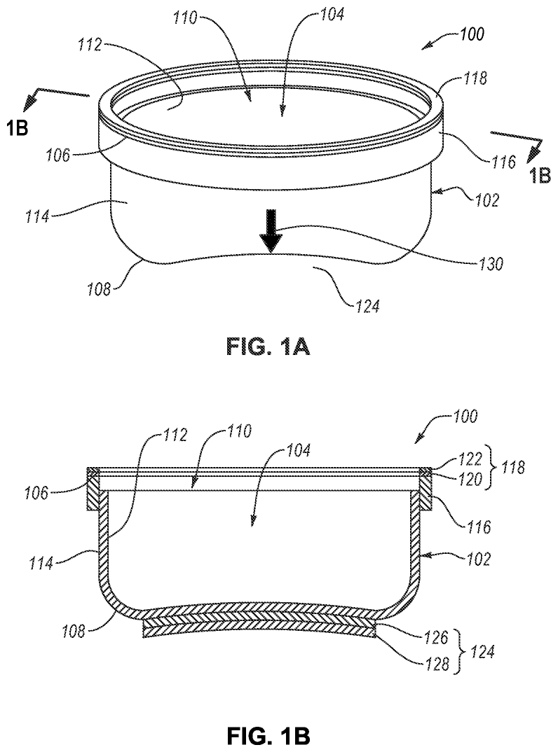

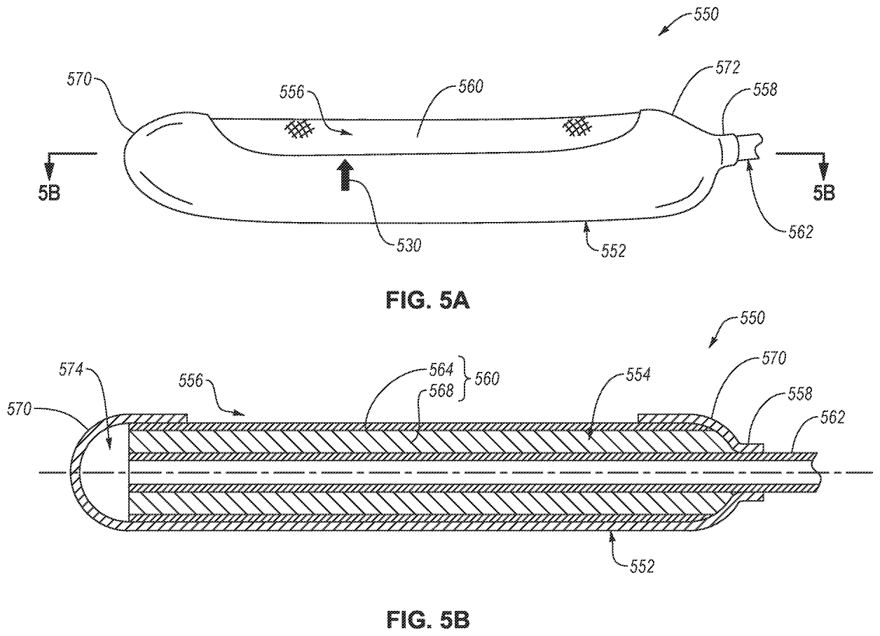

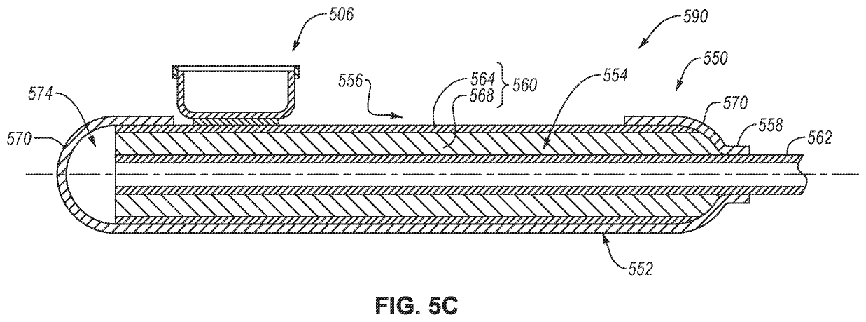

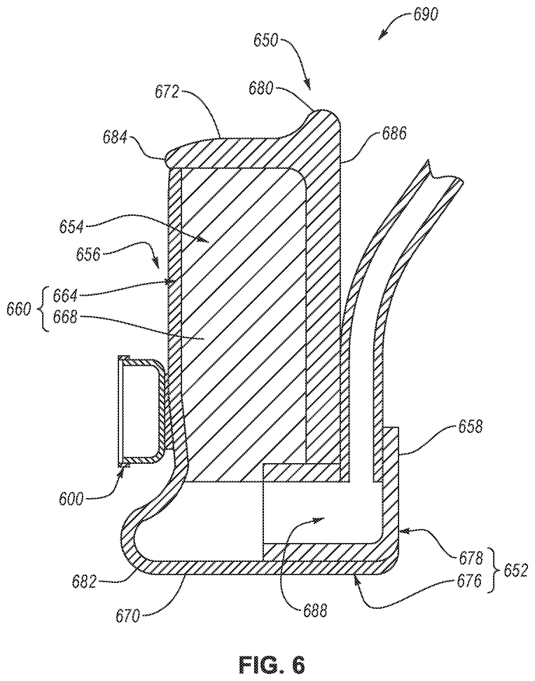

FIG. 1A is an isometric view of a menstrual container 100, according to an embodiment. FIG. 1B is a cross-sectional view of the menstrual container 100 taken along plane 1B-1B, according to an embodiment. The menstrual container 100 includes one or more fluid impermeable layers 102 defining a container chamber 104. The fluid impermeable layer 102 also includes a top side 106 configured to abut the vaginal region of an individual and a bottom side 108 opposite the top side 106. The top side 106 defines a bag opening 110 that allows menstrual fluids to flow into the container chamber 104. The bottom side 108 is configured to abut the fluid collection assembly (illustrated in FIGS. 5A-6)

The fluid impermeable layer 102 at least partially defines the container chamber 104 and the bag opening 110. For example, the interior surfaces 112 of the fluid impermeable layer 102 at least partially defines the chamber 104 within the menstrual container 100. The fluid impermeable layer 102 at least temporarily stores the menstrual fluids received by the menstrual container 100 in the container chamber 104. The container chamber 104 may exhibit a volume of about 5 milliliters (“ml”) or greater, such as about 5 ml to about 15 ml, about 10 ml to about 20 ml, about 15 ml to about 25 ml, about 20 ml to about 30 ml, about 25 ml to about 35 ml, about 30 ml to about 40 ml, about 35 ml to about 45 ml, about 40 ml to about 50 ml, about 45 ml to about 55 ml, about 50 ml to about 60 ml, about 55 ml to about 65 ml, about 60 ml to about 70 ml, about 65 ml to about 75 ml, about 70 ml to about 80 ml, about 75 ml to about 85 ml, about 80 ml to about 90 ml, about 85 ml to about 95 ml, or about 90 ml to about 100 ml. The volume of the container chamber 104 may be selected based on a number of factors. In an example, the volume of the container chamber 104 may be selected to be selected based the time period that the individual is expected to use the menstrual container 100 since the volume of the container chamber 104 may need to be increased as the expected period of time is increased. In an example, the volume of the container chamber 104 may be selected based on the expected volume of menstrual fluid discharge. For instance, the volume of the container chamber 104 may be increased when the expected volume of menstrual fluid discharge is high (e.g., during the first and second day of menstruation) and may be decreased when the expected volume of menstrual fluid discharge is expected to be small (e.g., during spotting or near the end of the period). In an example, the volume of the container chamber 104 is selected to be as small as possible since increasing the volume of the container chamber 104 may cause the portions of the fluid collection assembly adjacent to the menstrual container 100 to move away from the vaginal region. Moving the fluid collection assembly away from the vaginal region may increase the likelihood that the fluid collection assembly leaks bodily fluids.

The fluid impermeable layer 102 may be formed from any suitable fluid impermeable material(s), such a fluid impermeable polymer (e.g., silicone, polypropylene, polyethylene, polyethylene terephthalate, neoprene, a polycarbonate, etc.), a metal film, natural rubber, another suitable material, any other fluid impermeable material disclosed herein, or combinations thereof. As such, the fluid impermeable layer 102 substantially prevents the bodily fluids from passing through the fluid impermeable layer 102. At least one or more portions of at least an outer surface 114 of the fluid impermeable layer 102 may be formed from a soft and/or smooth material, thereby reducing chaffing.

In an embodiment, at least a portion of the fluid impermeable layer 102 is transparent. In such an embodiment, the transparent portions of the fluid impermeable layer 102 allows the individual using the menstrual container 100 or another individual (e.g., a medical practitioner) to easily view the container chamber 104. For example, the transparent portions of the fluid impermeable layer 102 allows such individuals to quickly determine the quantity of menstrual fluids in the container chamber 104, such as to quickly determine when the menstrual container 100 needs to be emptied or disposed of prior to the menstrual fluids leaking from the menstrual container 100.

In an embodiment, the fluid impermeable layer 102 includes a ring 116 forming at least a portion of the top side 106. The ring 116 exhibits a rigidity that is greater than the rest of the fluid impermeable layer 102. The ring 116 may exhibit a rigidity because at least one of the ring 116 exhibits a thickness that is greater than or a Young's modulus (i.e., modulus of elasticity) that is greater than the rest of the fluid impermeable layer 102. The ring 116 provides additional structure for the portions of the fluid impermeable layer 102 that define the bag opening 110 thereby preventing collapse of the bag opening 110 when the ring 116 is positioned between the labia folds of the individual. The ring 116 also allows the top side 106 of the fluid impermeable layer 102 to be molded or shaped to exhibit a shape that corresponds to a portion of the vaginal region (e.g., to the portion of the vaginal region defining the vaginal opening). In a particular embodiment, the ring 116 may be formed from silicone.

In an embodiment, the top side 106 of the fluid impermeable layer 102 (e.g., the ring 116) may define a generally circular bag opening 110. In an embodiment, the top side 106 may exhibit an elongated (e.g., a generally oval shaped) bag opening 110. The elongated opening 110 allows the bag opening 110 to extend along a greater length of the vaginal region while also fitting between the thighs of the individual.

The menstrual container 100 may be configured to be an external menstrual container 100 that is not inserted into the vaginal opening of the individual. Configuring the menstrual container 100 to be an external menstrual container decreases the likelihood that the menstrual container 100 causes an infection in the vaginal region and may allow the menstrual container 100 to be sterilized using less strenuously means (e.g., the menstrual container 100 does not need to be formed from a material that must withstand a strenuous sterilization process). Instead, the menstrual container 100 may be configured to be positioned such that at least one of the bag opening 110 is adjacent to the vaginal opening, at least a portion of the menstrual container 100 (e.g., at least the ring 116) is positioned between the labia folds, or the bag opening 110 is adjacent to the labia folds. Such positions of the menstrual container 100 allows the menstrual container 100 to receive at least some of the menstrual fluids while minimizing the likelihood that the menstrual fluids leak or are not received into the container chamber 104.

In an embodiment, when the menstrual container 100 is an external menstrual container, the menstrual container 100 may include a forward-facing adhesive structure 118 disposed on at least one of at least a portion of the top side 106 of the fluid impermeable layer 102 or at least a portion of the fluid impermeable layer 102 adjacent to the top side 106 (e.g., a lateral side of the ring 116). The forward-facing adhesive structure 118 may be configured to attach the fluid impermeable layer 102 to the vaginal region. Attaching the fluid impermeable layer 102 to the vaginal region prevents or at least inhibits the menstrual container 100 moving relative to the vaginal opening since movement of the menstrual container 100 relative to the vaginal opening may allow the menstrual fluids to flow around the menstrual container 100 instead of flowing through the bag opening 110 into the container chamber 104. Attaching the fluid impermeable layer 102 to the vaginal region also forms a seal that prevents or at least inhibits menstrual fluids flowing out of the container chamber 104. The forward-facing adhesive structure 118 includes an adhesive 120. The adhesive 120 may include any chemical or physical element that may attach the vaginal region. For example, the adhesive 120 may include a pressure-sensitive adhesive, a water activated adhesive, heat sensitive adhesive, nano tape (a.k.a., gecko tape), another Van der Waals attachment, or any other suitable adhesive. In an example, the forward-facing adhesive structure 118 also include a release liner 122 that protects the adhesive 120 and prevents the adhesive 120 becoming adhered to a structure other than the vaginal region of the individual. In an example, the forward-facing adhesive structure 118 may include a substrate (e.g., a polymer film, not shown) to which the adhesive 120 is attached. The substrate may be attached to the fluid impermeable layer 102.

The adhesive structure 118 may cause irritation to the vaginal region and/or may make detaching the menstrual container 100 from the vaginal region painful with some patients. As such, in an embodiment, the menstrual container 100 does not include the forward-facing adhesive structure 118. In such an embodiment, the menstrual container 100 may be held against or adjacent to the vaginal opening because, for example, at least one of the menstrual container 100 is positioned between the labia folds and the labia folds maintain the position of the menstrual container 100 or the fluid collection assembly to which the menstrual container 100 is abutted maintains the position of the menstrual container 100.

The bottom side 108 of the menstrual container 100 is configured to abut the fluid collection assembly. In an embodiment, the bottom side 108 may exhibit a shape that generally corresponds to the shape of the portions of the fluid collection assembly to which the bottom side 108 abuts. For example, as shown in FIGS. 5A-6, the bottom side 108 is configured to abut a convexly curved portion of a fluid collection assembly. As such, the bottom side 108 may exhibit a concave curve that generally corresponds to the convexly curved portion of the fluid collection assembly. In an embodiment, the bottom side 108 may be relatively flat.

In an embodiment, the menstrual container 100 may include a rearward-facing adhesive structure 124 disposed on at least one of at least a portion of the bottom side 108 of the fluid impermeable layer 102. The rearward-facing adhesive structure 124 may be configured to attach the fluid impermeable layer 102 to the fluid collection assembly. Attaching the fluid impermeable layer 102 to the fluid collection assembly prevents the menstrual container 100 moving relative to fluid collection assembly and allows the fluid collection assembly to maintain the position of the menstrual container 100.

The rearward-facing adhesive structure 124 includes an adhesive 126. The adhesive 126 may include any chemical or physical element that may attach the fluid collection assembly. In an embodiment, the adhesive 126 may be configured to be attached to the porous material of the fluid collection assembly (e.g., the porous material 560 of FIGS. 5A and 5B). In such an embodiment, the adhesive 126 may include a plurality of hooks (e.g., similar to the hooks of a Velcro attachment) that are able to grip the fibers of the porous material, a pressure sensitive adhesive configure to attach to the surface of the porous material substantially without flowing into the porous material, nano tape, another Van der Waals attachment, or any other suitable adhesive. In an example, the adhesive 126 is configured to allow the menstrual container 100 to be detached from the porous material substantially without damaging the adhesive 126 and the porous material. In such an example, the menstrual container 100 may be detached from the porous material for inspection, to empty the menstrual container 100, or for other purposes and then the menstrual container 100 may be reattached to the porous material for further use. In an example, the adhesive 126 does not include glue or other adhesive that may enter into and obstruct the pores of the porous material since, as previously discussed, obstructing the pores of the porous material may inhibit flow of the bodily fluids through the porous material. In an embodiment, the rearward-facing adhesive structure 124 is configured to be attached to the fluid impermeable barrier of the fluid collection assembly (e.g., the fluid impermeable barrier 552 of FIGS. 5A and 5B). In such an embodiment, the adhesive 126 may include a pressure-sensitive adhesive, nano tape, another Van der Waals attachment, glue, or any other suitable adhesive.

The rearward-facing adhesive structure 124 may include one or more structures in addition to the adhesive 126. In an example, the rearward-facing adhesive structure 124 includes a release liner 128 that protects the adhesive 126 and prevents the adhesive 126 becoming adhered to a structure other than the vaginal region of the individual. In an example, the rearward-facing adhesive structure 124 may include a substrate (e.g., a polymer film, not shown) to which the adhesive 126 is attached. The substrate may be attached to the fluid impermeable layer 102.

In an embodiment, the menstrual container 100 may include one or more markings 130. The markings 130 may include any structure that is visually distinguishable on the fluid impermeable layer 102 that are formed on or in the fluid impermeable layer 102. For example, the markings 130 may be printed on the fluid impermeable layer 102, formed from one or more recesses formed in the fluid impermeable layer 102, or formed from one or more protrusions extending from the fluid impermeable layer 102.

The markings 130 may be configured to facilitate positioning the menstrual container 100 on the fluid collection assembly. For example, the fluid collection assemblies illustrated in FIGS. 5A-6 include an elongated opening. Without the menstrual container 100 abutting the fluid collection assembly, a top portion of the elongated opening is positioned adjacent to the urethral opening and a bottom portion of the elongated opening (e.g., a portion closer to the distal end region than the top portion) is positioned adjacent to the urethral opening. When attaching the menstrual container 100 to such fluid collection assemblies, it is important to correctly position the menstrual container 100 on the fluid collection assembly. For example, positioning the menstrual container 100 to close to the top portion of the elongated opening may cause the menstrual container 100 to block or otherwise interfere with the fluid collection assembly receiving the bodily fluids from the urethral opening. Similarly, positioning the menstrual container 100 to far from the top portion of the elongated opening may make correct positioning of the fluid collection assembly on the individual difficult while maintaining the bag opening 110 adjacent to the vaginal opening. Failing to correctly position the fluid collection assembly may cause the fluid collection system to leak and/or move. Further, the markings 130 may facilitate aligning the menstrual container 100 with the vaginal region when the bag opening 110 exhibits an elongated shape.

In an embodiment, the markings 130 on the menstrual container 100 are configured to align with one or more features on the fluid collection assembly. For example, the markings 130 may be a line that is configured to be aligned with the boundary of the opening of the fluid collection assembly, an arrow (as shown) that is to be aligned with a terminal end of the opening or one or more markings formed on the fluid collection assembly, etc.

In an embodiment, the menstrual container 100 is reusable. The menstrual container 100 is reusable when the menstrual container 100 may be used two or more times and sterilized between each use without being damaged. In an embodiment, the menstrual container 100 may be configured for single use. For example, the menstrual container 100 may be configured for single use when at least a portion of the fluid impermeable layer 102 is formed from a film (e.g., exhibits a minimum thickness less than 0.5 mm).

In an embodiment, as illustrated, the menstrual container 100 exhibits a maximum width at or near the bag opening 110 and the width of the menstrual container 100 remains relatively constant from or near the top side 106 to the bottom side 108. In an embodiment, the menstrual container 100 exhibits a maximum width at a location that is spaced from the bag opening 110, such as at the bottom side 108 or at a location between the front side 106 and the bottom side 108. In such an embodiment, the container chamber 104 of the menstrual container 100 may exhibit a volume that is equal to or greater than the volume of the container chamber 104 of the menstrual container 100 exhibiting a maximum width at or near the top side 106 while preventing or minimizing the distance that the menstrual container 100 moves the fluid collection assembly away from the vaginal region.

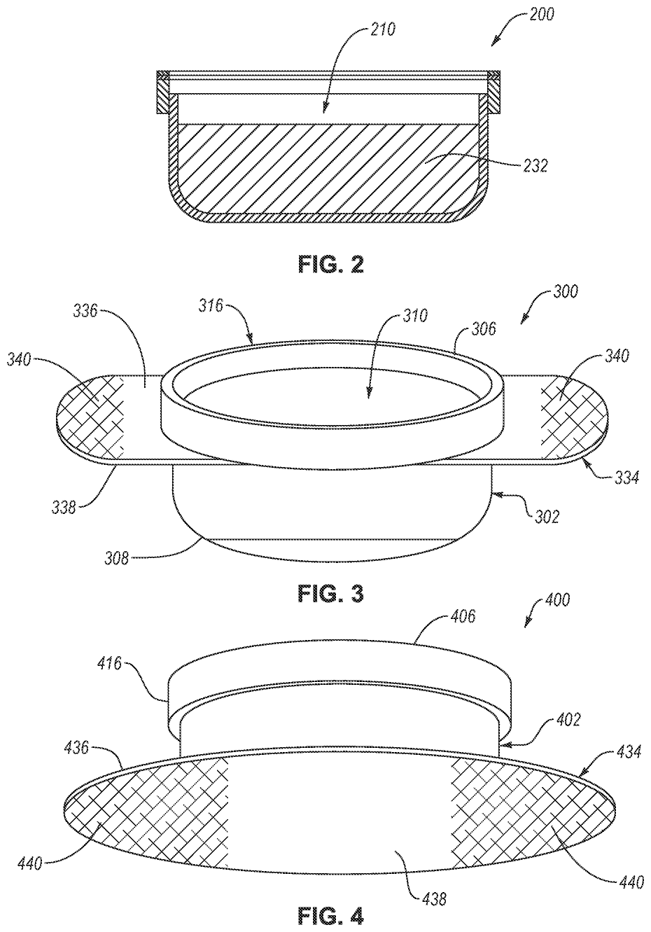

The container chamber 104 of the menstrual container 100 is substantially unoccupied which maximizes the quantity of menstrual fluids that are held therein, makes emptying the container chamber 104 easier, and may minimize the distance that the menstrual container moves the fluid collection assembly away from the vaginal region than if the container chamber 104 was at least partially occupied by a porous material. However, the substantially unoccupied container chamber 104 allows the menstrual fluids received therein to move therein which may cause the menstrual fluids to leak therefrom. As such, in some embodiments, the menstrual containers disclosed herein may include at least one porous material disposed in the container chamber 104. For example, FIG. 2 is a cross-sectional schematic of a menstrual container 200 including at least one porous material 232 disposed in the container chamber 204 therein, according to an embodiment. Except as otherwise disclosed herein, the menstrual container 200 may be the same or substantially similar to any of the menstrual containers disclosed herein.

The porous material 232 may include any material that may receive the menstrual fluids. For example, the porous material 232 may include fabric, gauze (e.g., a silk, linen, or cotton gauze), another soft fabric, another smooth fabric, a nonwoven material, a woven material (e.g., spun nylon fibers), a porous polymer (e.g., nylon, polyester, polyurethane, polyethylene, polypropylene, etc.) structure, an open cell foam, or two more different porous materials (e.g., a top layer and a bottom layer).

As previously discussed, the menstrual containers disclosed herein may include a structure that attaches the menstrual containers directly to the individual. For example, as discussed in more detail with regards to FIGS. 1A and 1B, the menstrual containers disclosed herein may include the forward-facing adhesive structure 118 that is configured to attach the menstrual container 100 to the vaginal region. However, it is noted that the menstrual containers disclosed herein may include structures other than or in addition to the forward-facing adhesive structure 118 that are configured to attach the menstrual container 100 directly to the individual. FIG. 3 is an isometric view of a menstrual container 300 that includes one or more wings 334 that are configured to directly attach the menstrual container 300 to the individual, according to an embodiment. Except as otherwise disclosed herein, the menstrual container 300 may be the same or substantially similar to any of the menstrual containers disclosed herein. For example, the menstrual container 300 may include a fluid impermeable layer 302 including a top side 306 defining a bag opening 310 and a bottom side 308. The fluid impermeable layer 302 may also include a ring 316.

As previously discussed, the menstrual container 300 includes one or more wings 334. The wings 334 are structures attached to and extending lateral (e.g., radially relative to the bag opening 310) away from the fluid impermeable layer 302. The wings 334 are configured to attach the menstrual container 300 to the individual. The wings 334 may extend from or near the top side 306 of the fluid impermeable layer 302. For example, when the fluid impermeable layer 302 includes the ring 316 (as shown), the wings 334 may extend from the portion of the ring 316 defining the top side 306, a lateral surface of the ring 316, or from a bottom edge of the ring 316 (e.g., an edge of the ring 316 opposite the top side 306).

The wings 334 include a front wing side 336 and a back wing side 338 opposite the front wing side 338. The front wing side 336 is the side of the wings 334 that face the individual (e.g., face the vaginal region and/or thighs of the individual) during use. For example, the front wing side 336 may face the same general direction as the top side 306 of the fluid impermeable layer 302. The wings 334 include a wing adhesive structure 340 (illustrated schematically on the wings 334 using cross-hatching) disposed on at least a portion of the front wing side 336. The wing adhesive structure 340 is configured to attach the wing 334 to the individual, such as to the vaginal region and/or the thighs of the individual. The wing adhesive structure 340 may be substantially similar to any of the adhesive structures disclosed herein. For example, the wing adhesive structure 340 includes an adhesive (e.g., any of the adhesives disclosed herein) and may optionally include other structures, such as a release liner or a substrate. In an example, the wing adhesive structure 340 includes an adhesive that is weaker than the adhesive that attaches the menstrual container 300 to the fluid collection assembly. The weaker adhesive of the wing adhesive structure 340 decreases the likelihood that detaching the menstrual container 300 from the individual causes injury or unnecessary pain to the individual and decreases the likelihood that significant movement of the fluid collection assembly (e.g., removing the fluid collection assembly from the individual) detaches the menstrual container 300 from the fluid collection assembly.

In an embodiment, the wing adhesive structure 340 covers all or substantially all of the front wing side 336. In such an embodiment, the wing adhesive structure 340 is attached to the vaginal region and, if the wings 334 are long enough, the thighs of the individual. In an embodiment, the wings adhesive structure 340 covers only a portion of the front wing side 336. For example, the wing adhesive structure 340 may only cover portions of the front wing side 336 that are spaced from the fluid impermeable layer 302. In such an example, spacing the wing adhesive structure 340 from the fluid impermeable layer 302 may decrease the percentage of the sensitive vaginal region that the wing 334 is attached to thereby decreasing the pain caused by detaching the wing 334 from the vaginal region. In an embodiment, the wing adhesive structure 340 is formed from two or more different adhesives having different adhesion strength. For example, the portions of the wing adhesive structure 340 that are configured to be attached to the vaginal region may exhibit a weaker adhesion strength that the portions of the wing adhesive structure 340 that are configured to be attached to the thighs of the individual. In such an example, the adhesive structure 340 allows the wing 334 to be strongly attached to the individual while minimizing any discomfort or pain caused by detaching the menstrual container 300 from the individual.

In an embodiment, as illustrated, the wings 334 do not surround the fluid impermeable layer 302 (e.g., do not surround the ring 316). In an embodiment, the wings 334 may completely surround the fluid impermeable layer 302 (e.g., completely surround the ring 316) which may facilitate forming a seal around the vaginal opening.

As previously discussed, the menstrual containers disclosed herein may include a structure that attaches the menstrual containers directly to the fluid collection assembly. For example, as discussed in more detail with regards to FIGS. 1A and 1B, the menstrual containers disclosed herein may include the rearward-facing adhesive structure 124 that is configured to attach the menstrual container 100 to the fluid collection assembly. However, it is noted that the menstrual containers disclosed herein may include structures other than or in addition to the rearward-facing adhesive structure 124 that are configured to attach the menstrual container 100 directly to the fluid collection assembly. FIG. 4 is an isometric view of a menstrual container 400 that includes one or more wings 434 that are configured to directly attach the menstrual container 400 to a fluid collection assembly, according to an embodiment. Except as otherwise disclosed herein, the menstrual container 400 may be the same or substantially similar to any of the menstrual containers disclosed herein. For example, the menstrual container 400 may include a fluid impermeable layer 402 including a top side 406 defining a bag opening 410 and a back side (obstructed by the wing 434). The fluid impermeable layer 402 may also include a ring 416.

As previously discussed, the menstrual container 400 includes one or more wings 434. The wings 434 are structures attached to and extending lateral (e.g., radially relative to the bag opening 410) away from the fluid impermeable layer 402. The wings 434 are configured to attach the menstrual container 400 to the fluid collection assembly. The wings 434 extend from or near the bottom side 408 of the fluid impermeable layer 402.

The wings 434 include a front wing side 436 and a back wing side 438 opposite the front wing side 436. The back wing side 438 is the side of the wings 434 that face the fluid collection assembly during use. For example, the back wing side 438 may face the same general direction as the bottom side 408 of the fluid impermeable layer 402. The wings 434 include a wing adhesive structure 440 (illustrated schematically on the wings 434 using cross-hatching) disposed on at least a portion of the back wing side 438. The wing adhesive structure 440 is configured to attach the wing 436 to the fluid collection assembly. The wing adhesive structure 440 may be substantially similar to any of the adhesive structures disclosed herein. For example, the wing adhesive structure 440 includes an adhesive (e.g., any of the adhesives disclosed herein) and may optionally include other structures, such as a release liner or a substrate.

In an embodiment, the wing adhesive structure 440 covers all or substantially all of the back wing side 438. In such an embodiment, the wing adhesive structure 440 may be attached to the porous material which, as previously discussed, may limit the types of adhesives that attach the wing adhesive structure 440 to the fluid collection assembly to prevent or limit a blockage of the pores by the adhesive. In an embodiment, the wings adhesive structure 440 covers only a portion of the back wing side 438. For example, the wing adhesive structure 440 may cover the lateral portions of the back wing side 438 (e.g., not the central portions). In such an example, the wing adhesive structure 440 may not be attached to the porous material of the fluid collection assembly thereby allowing the adhesive of the wing adhesive structure 440 to be selected from a larger variety of adhesives. In an embodiment, the wing adhesive structure 440 is formed from two or more different adhesives. For example, the portions of the wing adhesive structure 440 that are configured to be attached to the porous material may be selected from an adhesive that does not or substantially does not obstruct the pores of the porous material while the adhesive of the wing adhesive structure 440 that is not configured to be attached to the porous material may be formed from an adhesive that would, is attached to the porous material, would at least partially obstruct the pores of the porous material.

As previously discussed, the menstrual containers disclosed herein may be used with a fluid collection assembly. FIG. 5A is a side view of a fluid collection assembly 550, according to an embodiment. FIG. 5B is a cross-sectional schematic of the fluid collection assembly 550 taken along plane 5B-5B shown in FIG. 5A. The fluid collection assembly 550 includes a fluid impermeable barrier 552. The fluid impermeable barrier 552 defines a chamber 554, an opening 556, and a fluid outlet 558. The fluid collection assembly 550 also includes at least one porous material 560 disposed in and at least partially occupying the chamber 554.

The fluid impermeable barrier 552 at least partially defines a chamber 554 (e.g., interior region) and an opening 556. For example, the interior surface(s) of the fluid impermeable barrier 552 at least partially defines the chamber 554 within the fluid collection assembly 550. The fluid impermeable barrier 552 temporarily stores the bodily fluids in the chamber 554. The fluid impermeable barrier 552 may be formed of any suitable fluid impermeable material(s), such as a fluid impermeable polymer (e.g., silicone, polypropylene, polyethylene, polyethylene terephthalate, neoprene, polycarbonate, etc.), a metal film, natural rubber, another suitable material, any other fluid impermeable material disclosed herein, or combinations thereof. As such, the fluid impermeable barrier 552 substantially prevents the bodily fluids from passing through the fluid impermeable barrier 552. In an example, the fluid impermeable barrier 552 may be air permeable and fluid impermeable. In such an example, the fluid impermeable barrier 552 may be formed of a hydrophobic material that defines a plurality of pores. At least one or more portions of at least an outer surface of the fluid impermeable barrier 552 may be formed from a soft and/or smooth material, thereby reducing chaffing.

In some examples, the fluid impermeable barrier 552 may be tubular (ignoring the opening 556), such as substantially cylindrical (as shown), oblong, prismatic, or flattened tubes. During use, the outer surface of the fluid impermeable barrier 552 may contact the individual. The fluid impermeable barrier 552 may be sized and shaped to fit between the labia and/or the gluteal cleft between the legs of a female user.

The opening 556 provides an ingress route for bodily fluids to enter the chamber 554. The opening 556 may be defined by the fluid impermeable barrier 552 such as by an inner edge of the fluid impermeable barrier 552. For example, the opening 556 is formed in and extends through the fluid impermeable barrier 552, from the outer surface to the inner surface, thereby enabling bodily fluids to enter the chamber 554 from outside of the fluid collection assembly 550.

The opening 556 may be an elongated hole in the fluid impermeable barrier 552. For example, the opening 556 may be defined as a cut-out in the fluid impermeable barrier 552. The opening 556 may be located and shaped to be positioned adjacent to a female urethral opening. The opening 556 may have an elongated shape because the space between the legs of a female is relatively small when the legs of the female are closed, thereby only permitting the flow of the bodily fluids along a path that corresponds to the elongated shape of the opening 556 (e.g., longitudinally extending opening 556).

The fluid collection assembly 550 may be positioned proximate to the female urethral opening and the bodily fluids may enter the chamber 554 of the fluid collection assembly 550 via the opening 556. The fluid collection assembly 550 is configured to receive the bodily fluids into the chamber 554 via the opening 556. When in use, the opening 556 may have an elongated shape that extends from a first location below the urethral opening (e.g., at or near the anus or the vaginal opening) to a second location above the urethral opening (e.g., at or near the top of the vaginal opening or the pubic hair).

In some examples, the fluid impermeable barrier 552 may define a fluid outlet 558 sized to receive the conduit 562. The at least one conduit 562 may be disposed in the chamber 554 via the fluid outlet 558. The fluid outlet 558 may be sized and shaped to form an at least substantially fluid tight seal against the conduit 562 or the at least one tube thereby substantially preventing the bodily fluids from escaping the chamber 554.

In an embodiment, the fluid impermeable barrier 552 may include one or more markings 530. The markings 530 may include any structure that is visually distinguishable on the fluid impermeable barrier 552 that are formed on or in the fluid impermeable barrier 552. For example, the markings 530 may be printed on the fluid impermeable barrier 552, formed from one or more recesses formed in the fluid impermeable barrier 552, or formed from one or more protrusions extending from the fluid impermeable barrier 552.

The markings 530 may be configured to facilitate positioning a menstrual container on the fluid collection assembly. For example, as previously discussed, failing to correctly position the menstrual container on the fluid collection assembly 550 may cause the fluid collection assembly 550 to leak and/or move. In an embodiment, the markings 530 on the fluid impermeable barrier 552 are configured to align with one or more features on the menstrual container. For example, the markings 530 may be arrow that is configured to align with a marking on the menstrual container (e.g., the marking 130 of FIG. 1A).

In an embodiment, the porous material 560 may be configured to wick any bodily fluids away from the opening 556, thereby preventing the bodily fluids from escaping the chamber 554. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the porous material 560, such as not include adsorption of the bodily fluids into the fluid permeable support 568. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the porous material 560 (e.g., absorbency), such as less than about 30 wt % of the dry weight of the porous material 560, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the porous material 560. The porous material 560 may also wick the bodily fluids generally towards an interior of the chamber 554, as discussed in more detail below. In an embodiment, the porous material 560 may include at least one absorbent or adsorbent material.

In an embodiment, the porous material 560 may include the fluid permeable membrane 564 disposed in the chamber 554. The fluid permeable membrane 564 may cover at least a portion (e.g., all) of the opening 556. The fluid permeable membrane 564 may be composed to wick the bodily fluids away from the opening 556, thereby preventing the bodily fluids from escaping the chamber 554.

In an embodiment, the fluid permeable membrane 564 may include any material that may wick the bodily fluids. For example, the fluid permeable membrane 564 may include fabric, such as a gauze (e.g., a silk, linen, or cotton gauze), another soft fabric, another smooth fabric, a nonwoven material, or any of the other porous materials disclosed herein. Forming the fluid permeable membrane 564 from gauze, soft fabric, and/or smooth fabric may reduce chaffing caused by the fluid collection assembly 550.

The fluid collection assembly 550 may include the fluid permeable support 568 disposed in the chamber 554. The fluid permeable support 568 is configured to support the fluid permeable membrane 564 since the fluid permeable membrane 564 may be formed from a relatively foldable, flimsy, or otherwise easily deformable material. For example, the fluid permeable support 568 may be positioned such that the fluid permeable membrane 564 is disposed between the fluid permeable support 568 and the fluid impermeable barrier 552. As such, the fluid permeable support 568 may support and maintain the position of the fluid permeable membrane 564. The fluid permeable support 568 may include any material that may wick, absorb, adsorb, or otherwise allow fluid transport of the bodily fluids, such as any of the fluid permeable membrane materials disclosed herein above. For example, the fluid permeable membrane material(s) may be utilized in a more dense or rigid form than in the fluid permeable membrane 564 when used as the fluid permeable support 568. The fluid permeable support 568 may be formed from any fluid permeable material that is less deformable than the fluid permeable membrane 564. For example, the fluid permeable support 568 may include a porous polymer (e.g., nylon, polyester, polyurethane, polyethylene, polypropylene, etc.) structure or an open cell foam, such as spun nylon fiber. In some examples, the fluid permeable support 568 may include a nonwoven material. In some examples, the fluid permeable support 568 may be formed from a natural material, such as cotton, wool, silk, or combinations thereof. In such examples, the material may have a coating to prevent or limit absorption of fluid into the material, such as a water repellent coating. In some examples, the fluid permeable support 568 may be formed from fabric, felt, gauze, or combinations thereof.

In some examples, the fluid permeable membrane 564 may be optional. For example, the porous material 560 may include only the fluid permeable support 568. In some examples, the fluid permeable support 568 may be optionally omitted from the fluid collection assembly 550. For example, the porous material 560 may only include the fluid permeable membrane 564.

In an embodiment, at least a portion of the porous material 560 (e.g., one or more of the fluid permeable membrane 564 or, more preferably, the fluid permeable support 568) may be hydrophobic. The porous material 560 may be hydrophobic when the porous material 560 exhibits a contact angle with water (a major constituent of bodily fluids) that is greater than about 90°, such as in ranges of about 90° to about 120°, about 105° to about 135°, about 120° to about 150°, about 135° to about 175°, or about 150° to about 180°. The hydrophobicity of the porous material 560 may limit absorption, adsorption, and solubility of the bodily fluids in the porous material 560 thereby decreasing the amount of bodily fluids held in the porous material 560. In an embodiment, at least a portion of the porous material 560 is hydrophobic or hydrophilic. In an embodiment, the fluid permeable support 568 is more hydrophobic (e.g., exhibits a larger contact angle with water) than the fluid permeable membrane 564. The lower hydrophobicity of the fluid permeable membrane 564 may help the porous material 560 receive the bodily fluids from the urethral opening while the hydrophobicity of the fluid permeable support 568 limits the bodily fluids that are retained in the porous material 560.

The conduit 562 may be at least partially disposed in the chamber 554. The conduit 562 may be used to remove the bodily fluids from the chamber 554. The conduit 562 may be operably coupled to a vacuum source, such as a vacuum pump for withdrawing fluid from the chamber 554 through the conduit 562. For example, the conduit 562 may extend into the fluid impermeable barrier 552 from the proximal end region 572 and may extend to the distal end region 570 to a point proximate to the reservoir 574 therein such that the inlet is in fluid communication with the reservoir 574. The conduit 562 fluidly couples the chamber 554 with the fluid storage container (not shown) or the vacuum source (not shown).

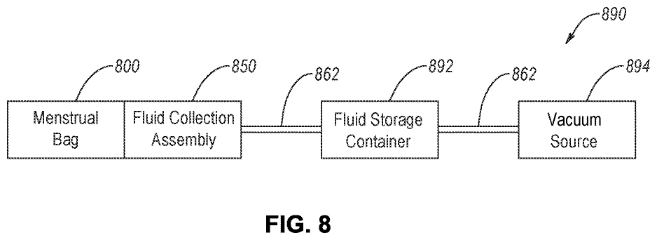

The conduit 562 is configured to fluidly couple (e.g., directly or indirectly) the vacuum source (not shown) to the chamber 554 (e.g., the reservoir 574). As the vacuum source (e.g., vacuum source 894 of FIG. 8) applies a vacuum/suction in the conduit 562, the bodily fluids in the chamber 554 (e.g., at the distal end region 570 such as in the reservoir 574) may be drawn into the conduit 562 and out of the fluid collection assembly 550 via the conduit 562. In some examples, the conduit 562 may be frosted or opaque (e.g., black) to obscure visibility of the bodily fluids therein.

The porous material 560 may at least substantially completely fill the portions of the chamber 554 that are not occupied by the conduit 562. In some examples, the porous material 560 may not substantially completely fill the portions of the chamber 554 that are not occupied by the conduit 562. In such an example, the fluid collection assembly 550 includes the reservoir 574 disposed in the chamber 554.

The reservoir 574 is a substantially unoccupied portion of the chamber 554. The reservoir 574 may be defined between the fluid impermeable barrier 552 and one or both of the fluid permeable membrane 564 and fluid permeable support 568. The bodily fluids that are in the chamber 554 may flow through the fluid permeable membrane 564 and/or fluid permeable support 568 to the reservoir 574. The reservoir 574 may retain of the bodily fluids therein.

The bodily fluids that are in the chamber 554 may flow through the fluid permeable membrane 564 and/or fluid permeable support 568 to the reservoir 574. The fluid impermeable barrier 552 may retain the bodily fluids in the reservoir 574. While depicted in the distal end region 570, the reservoir 574 may be located in any portion of the chamber 554 such as the proximal end region 572. The reservoir 574 may be located in a portion of the chamber 554 that is designed to be located in a gravimetrically low point of the fluid collection assembly when the fluid collection assembly is worn. The conduit 562 may extend into the fluid reservoir 574 and/or the inlet of the conduit 562 may be positioned adjacent to or proximate to the fluid reservoir 574.

In some examples (not shown), the fluid collection assembly 550 may include multiple reservoirs, such as a first reservoir that is located at the portion of the chamber 554 closest to the inlet of the conduit 562 (e.g., distal end region 570) and a second reservoir that is located at the portion of the of the chamber 554 that is at or near proximal end region 572). In another example, the fluid permeable support 568 is spaced from at least a portion of the conduit 562, and the reservoir 574 may be the space between the fluid permeable support 568 and the conduit 562.

FIG. 5C is a cross-sectional schematic of a fluid collection system 590 including the fluid collection assembly 550 with a menstrual container 500 attached thereto, according to an embodiment. The menstrual container 500 may be the same or substantially similar to any of the menstrual containers disclosed herein. In an embodiment, as illustrated, the menstrual container 500 is attached to a portion of the porous material 560 that is closer to the distal end region 570 of the fluid collection assembly 550 than a proximal end region 572. In such an example, the menstrual container 500 may be positioned adjacent to the vaginal opening of the individual, the portions of the porous material 560 more proximate to the proximal end region 572 may be positioned adjacent to the urethral opening, and the conduit 562 may extend from the fluid collection assembly 550 towards the stomach of the individual which has been shown to more comfortable and better at preventing leaks. In an embodiment, not shown, the menstrual container 500 may be attached to a portion of the fluid impermeable barrier 552 between the opening 556 and the distal most portion of the fluid impermeable barrier 552.

FIG. 5D is a cross-sectional view of the fluid collection system 590 during use, according to an embodiment. As shown, the fluid collection system 590 may be positioned adjacent to a vaginal region of an individual 571 such that the menstrual container 506 is disposed adjacent to or at least partially within a vaginal opening 573. For example, as illustrated, the menstrual container 506 is positioned over the vaginal opening 573. However, it is noted that the menstrual container 506 may be at least partially disposed in the vaginal opening 573, such as between the labia folds or at least partially disposed in the vaginal channel 575. Such positioning of the menstrual container 506 allows the menstrual container 506 to receive at least some (e.g., substantially all) of the menstrual fluids dispensed from the vaginal opening 573. The fluid collection system 590 is also positioned such that the opening 556 and the porous material 560 of the fluid collection assembly 550 are positioned adjacent to the urethral opening 577. As such, urine or other bodily fluids dispense from the urethral opening 577 flow through the opening 556 and is received into the porous material 560.

FIG. 6 is a cross-sectional schematic of a fluid collection system 690 including a fluid collection assembly 650 and a menstrual container 600, according to an embodiment. The fluid collection assembly 650 is example of a female fluid collection assembly for receiving and collecting bodily fluids from a female. Except as otherwise disclosed herein, the menstrual container 600 and the fluid collection assembly is the same as or substantially similar to any of the menstrual containers and the fluid collection assemblies disclosed herein, respectively.

The fluid collection assembly 650 includes a fluid impermeable barrier 652 that is the same or similar to any of the fluid impermeable barriers disclosed herein in one or more aspects. The fluid impermeable barrier 652 at least defines a chamber 654, at least one opening 656, and a fluid outlet 658. The fluid collection assembly 650 also includes at least one porous material 660 positioned within the chamber 654. The porous material 660 may be the same or similar to any of the porous materials disclosed herein in one or more aspects. The fluid collection assembly 650 may also include at least one conduit 662 partially positioned within the fluid outlet 658 that is configured to remove one or more bodily fluids from the chamber 654. The conduit 662 may not extend through the porous material 660.

In an embodiment, the fluid impermeable barrier 652 may include a shell 676 and a connector piece 678 secured to the shell 676. The shell 676 of the fluid collection assembly 650 includes a proximal end region 672, a distal end region 670 opposite the proximal end region 672, a front side 684, and a back side 686 opposite the front side 684. Generally, during use, the distal end region 670 is closer to the gluteal cleft of the individual than the proximal end region 672 and the front side 684 generally faces the vaginal region of the individual. The shell 676 may be formed from silicone, neoprene, a thermoplastic elastomer, or other fluid impermeable material.

In an embodiment, the shell 676 includes one or more flanges. The flanges may provide more locations for underwear or other clothing to contact and press against the fluid collection assembly 650 which may facilitate securing the fluid collection assembly 650 to the vaginal region of the individual and may improve patient comfort. In an embodiment, the flanges may include at least one of an upper flange 680 forming the proximal end region 672 and a bottom flange opposite the upper flange 680 that forms the distal end region 670.

The flanges of the body may extend from the rest of the shell 676 by a distance that is about 1 mm or greater, about 1 mm or greater, about 3 mm or greater, about 4 mm or greater, about 5 mm or greater, about 6 mm or greater, about 7.5 mm or greater, about 1 cm or greater, about 1.25 cm or greater, about 1.5 cm or greater, about 1 cm or greater, about 1.5 cm or greater, about 3 cm or greater, about 4 cm or greater, about 5 cm or greater, or in ranges of about 1 mm to about 3 mm, about 1 mm to about 4 mm, about 3 mm to about 5 mm, about 4 mm to about 6 mm, about 5 mm to about 7.5 mm, about 6 mm to about 1 cm, about 7.5 mm to about 1.25 cm, about 1 cm to about 1.5 cm, about 1.25 cm to about 1 cm, about 1.5 cm to about 1.5 cm, about 1 cm to about 3 cm, about 1.5 cm to about 4 cm, or about 3 cm to about 5. The distance that the flanges extend from the rest of the shell 676 may be selected based on the expected size of the vaginal region of the individual (e.g., larger flanges for a larger vaginal region) or the expected rotational forces applied to the fluid collection assembly 650 during use. In some examples, at least some of the flanges may extend further from the rest of the shell 676 that other flanges.

In an embodiment, the shell 676 may include a sump 682 at or near the distal end region 670. The sump 682 may extend outwardly from the front side 684 of the shell 676. During use, the sump 682 is configured to be at, near, or otherwise in fluid communication with a gravimetric low point of the porous material 660. For example, the sump 682 may receive a portion of the porous material 660 therein. The sump 682 may receive at least some of the bodily fluids that are received by the porous material 660. The sump 682 may prevent or at least inhibit bodily fluids from leaking from the fluid collection assembly 650. The sump 682 may include at least a portion of the connector piece 678 at least partially disposed therein.

In an embodiment, the shell 676 may define a recess that is configured to receive a conduit 662. The recess may extend from or near the proximal end region 672 to or near the distal end region 670 thereby allowing the conduit 662 to extend from or near the individual's abdominal region to the connector piece 678. In an embodiment, the recess may be configured such that the shell 676 encloses and/or abuts less than 60% of a circumference of the conduit 662, thereby allowing the conduit 662 to freely enter and leave the recess during use. Allowing the conduit 662 to freely enter and leave the recess may facilitate positioning of the fluid collection assembly 650 such that the porous material 660 is adjacent to the vaginal region even when the conduit 662 is bending away from the vaginal region. Also, allowing the conduit 662 to freely enter and leave the recess may increase the likelihood that movement of the conduit 662 does not move the porous material 660 relative to the vaginal region since movement of the porous material 660 may cause leaking. In an embodiment, at least a portion of the recess may be configured such that the shell 676 encloses and/or abuts more than 50% (e.g., 51% to about 55%, about 53% to about 57%, or about 55% to about 60%) of the circumference of the conduit 662. Enclosing more than 60% of the circumference of the conduit 662 may more securely attach the conduit 662 to the shell 676 and may allow the conduit 662 to provide additional structure to the shell 676. The percentage of the conduit 662 enclosed and/or abutted by the shell 676 may be selected such that the inherent elasticity of the shell 676 and the conduit 662 allows the conduit 662 to be easily snapped into and out of the recess. As such, the conduit 662 may be removed from the recess to facilitate positioning the porous material 660 adjacent to the vaginal region or when the conduit 662 is moved.

As previously discussed, the fluid impermeable barrier 652 includes a connector piece 678 that is attached to (e.g., with an adhesive, welding, interference fit, etc.) or integrally formed with the shell 676. The connector piece 678 is positioned at or near the distal end region 670 of the shell 676 which allows the connector piece 678 to receive bodily fluids that flow to the gravimetric low point of the porous material 660. In an embodiment, a portion of the connector piece 678 may be positioned in the sump 682 of the shell 676. In an embodiment, not shown, the connector piece 678 may form the sump 682 instead of the shell 676.

The connector piece 678 is configured to be connected to the conduit 662. As such, the connector piece 678 may define the fluid outlet 658 which configured to be attached to or otherwise in fluid communication with the conduit 662. The fluid outlet 658 may be positioned adjacent to the back side 686 of the fluid impermeable barrier 652. The connector piece 678 may also define a channel 688 (e.g., tube) configured to allow the porous material 660 and the sump 682 to be in fluid communication with the conduit 662.

In an embodiment, at least a portion of the connector piece 678 may exhibit a rigidity that is greater than the shell 676. The increased rigidity of the connector piece 678 relative to the shell 676 may facilitate attachment of the conduit 662 to the connector piece 678. In an example, the connector piece 678 may exhibit a rigidity that is greater than the shell 676 when the connector piece 678 is formed from a material exhibiting at least one of a greater Young's modulus (i.e., modulus of elasticity), yield strength, or ultimate tensile strength than a material that forms the shell 676. In an example, the connector piece 678 may exhibit a rigidity that is greater than the shell 676 when the connector piece 678 exhibits a thickness that is greater than the shell 676.

In an embodiment, as illustrated, the menstrual container 600 is attached to a portion of the porous material 660 that is closer to the distal end region 670 of the fluid collection assembly 650 than a proximal end region 672. In such an example, the menstrual container 600 may be positioned adjacent to the vaginal opening of the individual, the portions of the porous material 660 more proximate to the proximal end region 672 may be positioned adjacent to the urethral opening, and the conduit 662 may extend from the fluid collection assembly 650 towards the stomach of the individual which has been shown to more comfortable and better at preventing leaks. In an embodiment, not shown, the menstrual container 600 may be attached to a portion of the fluid impermeable barrier 652 between the opening 656 and the distal most portion of the fluid impermeable barrier 652.

Further examples of fluid collection assemblies that are configured to receive bodily fluids from a female urethral opening are disclosed in U.S. Pat. No. 10,226,376 filed on Jun. 1, 2017, U.S. Pat. No. 10,390,989 filed on Sep. 8, 2016, U.S. Pat. No. 10,952,889 filed on Jun. 2, 2016, PCT Patent Application No. PCT/US2021/026607 filed on Apr. 10, 2020, U.S. patent application Ser. No. 17/597,673 filed on Jun. 16, 2020, and U.S. Provisional Patent Application No. 63/208,262 filed on Jun. 8, 2021, the disclosure of each of which is incorporated herein, in its entirety, by this reference.

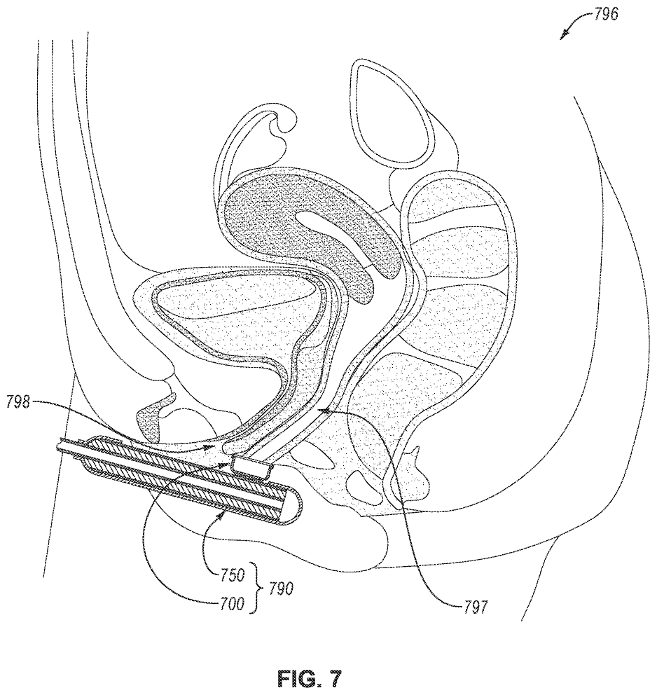

FIG. 7 is a cross-sectional schematic view of a fluid collection system 790 disposed on a female individual 796, according to an embodiment. Except as otherwise disclosed herein, the fluid collection system 790 may be the same or substantially similar to any of the fluid collection systems disclosed herein. For example, the fluid collection system 790 may include a fluid collection assembly 750 and a menstrual container 700 that are the same as or substantially similar to any of the fluid collection assemblies and menstrual containers disclosed herein, respectively. For illustrated purposes, the fluid collection system 790 is illustrates as being substantially the same as the fluid collection system 590 illustrated in FIG. 5C.

The fluid collection system 790 may be positioned on the individual 796 such that the menstrual container 700 is positioned adjacent to the vaginal opening 797. Such a position of the menstrual container 700 allows the menstrual container 700 to receive at least some of the blood and other menstrual fluids discharged from the vaginal opening 797. In other words, the menstrual container 700 may prevent the blood and other menstrual fluids from entering the porous material 760. The fluid collection assembly 750 is positioned on the individual 796 such that the opening thereof is positioned adjacent to the urethral opening 798 of the individual thereby allowing the fluid collection assembly 750 to receive at least some of the bodily fluids discharged from the urethral opening 798.

FIG. 8 is a block diagram of a fluid collection system 890 for fluid collection, according to an embodiment. The fluid collection system 890 includes a menstrual container 800, a fluid collection assembly 850, a fluid storage container 892, and a vacuum source 894. The menstrual container 800 and the fluid collection assembly 850 may be the same or substantially similar to any of the menstrual containers and the fluid collection assemblies disclosed herein, respectively. The fluid collection assembly 850, the fluid storage container 892, and the vacuum source 894 may be fluidly coupled to each other via one or more conduits 862. For example, fluid collection assembly 850 may be operably coupled to one or more of the fluid storage container 892 or the vacuum source 894 via the conduit 862. The bodily fluids collected in the fluid collection assembly 850 may be removed from the fluid collection assembly 850 via the conduit 862 which protrudes into the fluid collection assembly 850. For example, an inlet of the conduit 862 may extend into the fluid collection assembly 850, such as to a reservoir therein. The outlet of the conduit 862 may extend into the fluid collection assembly 850 or the vacuum source 894. Suction force may be introduced into the chamber of the fluid collection assembly 850 via the inlet of the conduit 862 responsive to suction (e.g., vacuum) force applied at the outlet of the conduit 862.

The suction force may be applied to the outlet of the conduit 862 by the vacuum source 894 either directly or indirectly. The suction force may be applied indirectly via the fluid storage container 892. For example, the outlet of the conduit 862 may be disposed within the fluid storage container 892 and an additional conduit 862 may extend from the fluid storage container 892 to the vacuum source 894. Accordingly, the vacuum source 894 may apply suction to the fluid collection assembly 850 via the fluid storage container 892. The suction force may be applied directly via the vacuum source 894. For example, the outlet of the conduit 862 may be disposed within the vacuum source 894. An additional conduit 862 may extend from the vacuum source 894 to a point outside of the fluid collection assembly 850, such as to the fluid storage container 892. In such examples, the vacuum source 894 may be disposed between the fluid collection assembly 850 and the fluid storage container 892.

The fluid storage container 892 is sized and shaped to retain bodily fluids therein. The fluid storage container 892 may include a bag (e.g., drainage bag), a bottle or cup (e.g., collection jar), or any other enclosed container for storing bodily fluids such as urine. In some examples, the conduit 862 may extend from the fluid collection assembly 850 and attach to the fluid storage container 892 at a first point therein. An additional conduit 862 may attach to the fluid storage container 892 at a second point thereon and may extend and attach to the vacuum source 894. Accordingly, a vacuum (e.g., suction) may be drawn through fluid collection assembly 850 via the fluid storage container 892. Bodily fluids, such as urine, may be drained from the fluid collection assembly 850 using the vacuum source 894.

The vacuum source 894 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 894 may provide a vacuum or suction to remove bodily fluids from the fluid collection assembly 850. In some examples, the vacuum source 894 may be powered by one or more of a power cord (e.g., connected to a power socket), one or more batteries, or even manual power (e.g., a hand operated vacuum pump). In some examples, the vacuum source 894 may be sized and shaped to fit outside of, on, or within the fluid collection assembly 850. For example, the vacuum source 894 may include one or more miniaturized pumps or one or more micro pumps. The vacuum sources 894 disclosed herein may include one or more of a switch, a button, a plug, a remote, or any other device suitable to activate the vacuum source 894.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1-13. (canceled)

14. A fluid collection system, comprising:
a fluid collection assembly including:
a fluid impermeable barrier at least defining a chamber, an opening, and a fluid outlet; and
at least one porous material disposed in the chamber and extending across the opening; and
external menstrual container including:
at least one fluid impermeable layer defining a container chamber, the at least one fluid impermeable layer separate from the fluid collection assembly, the external menstrual container including:
a top side configured to abut a vaginal region of an individual, the top side defining a bag opening configured to allow menstrual fluids to flow from the vaginal region into the container chamber; and
a bottom side opposite the top side, the bottom side at least one of configured to be attached to the fluid collection assembly, attached to the fluid collection assembly, or abutting the fluid collection assembly; and
wherein the external menstrual container prevents or inhibits menstrual fluids from entering the at least one porous material.

15. The fluid collection system of claim 14, wherein:
the fluid impermeable barrier includes one or more markings thereon that facilitate attachment of the external menstrual container to the fluid collection assembly; and
the external menstrual container includes one or more markings, and wherein the one or more markings of the external menstrual container are configured to aligned with the one or more markings of the fluid impermeable barrier when the external menstrual container is attached to the fluid collection assembly.

16. (canceled)

17. The fluid collection system of 14, wherein the external menstrual container is not attached to the fluid collection assembly.

18. The fluid collection system of 14, wherein the external menstrual container is attached to the at least one porous material.

19. The fluid collection system of 14, wherein the external menstrual container is attached to the fluid impermeable barrier.

20. The fluid collection system of 14, wherein the fluid impermeable barrier includes a proximal end region defining the fluid outlet and a distal end region opposite the proximal end region, the external menstrual container attached to the fluid collection assembly at a location that is closer to the distal end region than to the proximal end region.

21. The fluid collection system of claim 14, wherein the at least one fluid impermeable layer includes a ring forming the top side and defining the container opening, the ring exhibit a rigidity that is greater than a rigidity of a remainder of the at least one fluid impermeable barrier.

22. The fluid collection system of claim 14, wherein at least a portion of the at least one fluid impermeable layer is at least partially transparent.

23. The fluid collection system of claim 14, wherein the top side includes an adhesive configured to chemically or physically attach the top side to the vaginal region.

24. The fluid collection system of claim 14, further comprising an adhesive on the bottom side of the at least one fluid impermeable layer, the adhesive configured to attach the bottom side to the fluid collection assembly.

25. The fluid collection system of claim 24, wherein the adhesive lacks glue.

26. The fluid collection system of claim 24, wherein the adhesive includes a plurality of hooks.

27. The fluid collection system of claim 14, wherein the fluid impermeable layer includes one or more markings thereon configured to facilitate correct attachment of the external menstrual container to the fluid collection assembly.

28. The fluid collection system of claim 14, further comprising one or more wings extending from or near at least a portion of the portions of the top side of the fluid impermeable layer.

29. The fluid collection system of claim 28, wherein the one or more wings include a front wing side and a back wing side opposite the front wing side, at least a portion of the front wing side facing the vaginal region when the top side of the fluid impermeable layer abuts the vaginal region, at least a portion of the front wing side including an adhesive.

30. The fluid collection system of claim 14, further comprising one or more wings extending from or near at least a portion of the portions of the bottom side of the fluid impermeable layer, the one or more wings including a front wing side and a back wing side opposite the front wing side, at least a portion of the front wing side facing the vaginal region when the top side of the fluid impermeable layer abuts the vaginal region, at least a portion of the back wing side including an adhesive.

31. The fluid collection system of claim 14, wherein the container chamber is substantially unoccupied.

32. The fluid collection system of claim 14, further comprising at least one porous material disposed in the container chamber.

33. A method of forming a fluid collection system, the method comprising:
positioning an external menstrual container to cause a bottom side of the external menstrual container to abut or attach to a fluid collection assembly;
wherein the fluid collection assembly includes:
a fluid impermeable barrier at least defining a chamber, an opening, and a fluid outlet; and
at least one porous material disposed in the chamber and extending across the opening; and
wherein the external menstrual container is separate from the fluid collection assembly, the external menstrual container including:
at least one fluid impermeable layer defining a container chamber, the at least one fluid impermeable layer including:
a top side configured to abut a vaginal region of an individual, the top side defining a bag opening configured to allow menstrual fluids to flow from the vaginal region into the container chamber; and
the bottom side opposite the top side; and
wherein the external menstrual container prevents or inhibits menstrual fluids from entering the at least one porous material.

34. A method of using a fluid collection system, the method comprising:
positioning a bag opening of an external menstrual container adjacent to a vaginal opening of an individual and an opening of a fluid collection assembly adjacent to a urethral opening of the individual;
the fluid collection assembly including:
a fluid impermeable barrier at least defining a chamber, the opening, and a fluid outlet; and
at least one porous material disposed in the chamber and extending across the opening; and
the external menstrual container including:
a top side configured to abut a vaginal region of an individual, the top side defining a bag opening configured to allow menstrual fluids to flow from the vaginal region into the container chamber; and
a bottom side opposite the top side, the bottom side at least one of configured to be attached to the fluid collection assembly, attached to the fluid collection assembly, or abutting the fluid collection assembly;
wherein the external menstrual container is separate from the fluid collection assembly; and
wherein the external menstrual container prevents or inhibits menstrual fluids from entering the at least one porous material.
